(19) United States
(12) Patent Application Publication
(10) Pub. No.: US 2021/0298434 A1
(43) Pub. Date: Sep. 30, 2021

(54) MAGNETIC DERMAL ADHESIVES, ACCESSORIES, AND RELATED METHODS

(71) Applicant: LashLiner, Inc., Woodinville, WA (US)

(72) Inventor: Laura A. Hunter, Mill Creek, WA (US)

(21) Appl. No.: 17/341,787

(22) Filed: Jun. 8, 2021

Related U.S. Application Data
(62) Division of application No. 16/359,494, filed on Mar. 20, 2019.
(60) Provisional application No. 62/646,144, filed on Mar. 21, 2018.

(51) Int. Cl. A44C 15/00

(52) U.S. Cl. CPC A44C 15/0065; A44C 17/005; A44C 15/005

(57) ABSTRACT

Various magnetic dermal adhesives and magnetically attachable personalizable accessories of the present disclosure are provided comprising a load-bearing magnetic dermal adhesive incorporating magnetic particles and a magnetic personalizable accessory incorporating one or more magnetic elements exhibiting high magnetic coercivity and positioned at least at the base of the magnetic personalizable accessory enabling their interaction. These magnetically complementary products provide a convenient way for reversibly attaching/detaching/interchanging various magnetic personalizable accessories to interact with consumers' skin so that they can be (a) “re-useable” saving money, time, and resources; (b) can be arranged in various ways (worn singly or grouped together in an infinite number of unique arrangements); (c) can provide a broad range of options for creative self-expression/personal style; (d) can be interchangeably attached to achieve multiple variations; (e) can be conveniently swapped/exchanged from one user to another (f) can be stacked or superimposed together into various creative arrangements; (g) can be utilized for enhancing the experience of various personal and professional situations; and many more benefits that can be uniquely crafted by each consumer according to his/her particular performance requirements.

### CROSS-REFERENCE TO RELATED SPECIFICATION

This application claims the benefit of provisional application No. 62/646,144, filed on Mar. 21, 2018, the contents of which are incorporated herein by reference.

### TECHNICAL FIELD

The present disclosure relates to various load-bearing “magnetic dermal adhesives” for magnetically attaching a range of style-enhancing “magnetic personalizable accessories” to various skin surfaces of the face and body for aesthetic/fashionable enhancement and/or entertainment/amusement. Methods for making and using the disclosed load-bearing magnetic dermal adhesives for magnetically and reversibly attaching/detaching one or more “magnetic personalizable accessories” are also provided.

### BACKGROUND OF THE INVENTION

The necessity and desire to establish personal branding/personal expression by utilizing various personalizable accessories are increasingly appreciated by a diversity of global consumers. Some of these consumers are professional performers, including dancers, theatre actors, studio actors, comedians, musicians, magicians, acrobats, models, and other performers interested in intentionally altering their “stage” appearances quite frequently. Another large category of consumers includes children and adults who enjoy changing their appearances for special occasions/situations, such as holiday festivities, school events, social events, personal celebrations, weddings, graduations, birthdays, and many social activities where altering one's appearance is highly desirable and joyously entertaining.

Traditionally, cosmetics can be applied to faces in order to achieve desirable aesthetic effects, including the ability to change the perception of one's age, gender, skin pigmentation, and various emotional states. The entertainment-related industries are demonstrating the extreme possibilities in pushing the boundaries of cosmetic/costume manipulations to completely alter one's appearance using various cosmetics, dermal paints, and sculpting materials in creating fantasy characters and creatures of interest.

On another level, many consumers are increasingly choosing to alter their natural skin by tattooing various words/motifs on their faces and bodies. In the US alone, the tattoo and body art/piercing industries are gaining steady growth in interest, especially among Millennials. The tattoo industry (including body art/piercing) also includes tattoo-removal products and services generating a combined market estimated to be worth $3 billion in 2018. However, it appears that many consumers who once enjoyed their “permanent” tattoos often change their minds about the selected tattoo designs and/or desire to remove them entirely. Such unsatisfied consumers often pay thousands for professionals to remove “outdated/outlived/disfiguring” tattoos, involving time-consuming trips to removal clinics (requiring multiple sessions) and substantial physical and emotional pain.

There is an unmet need for more innovative, fashion-forward, style-enhancing products that can be highly personalized by consumers to enable a broad range of options in achieving unlimited personal branding/personal expression.

### SUMMARY

In several embodiments of the present invention, various systems for magnetically attaching a range of “personalizable accessories” to various skin surfaces of the face and body for “cosmetic/aesthetic” enhancement are provided, comprising: (a) a load-bearing magnetic dermal adhesive adherable to a skin surface and incorporating magnetic particles compatible with a load-bearing structural adhesive; and (b) a magnetically attachable “personalizable accessory” incorporating one or more magnetic elements exhibiting high magnetic coercivity and positioned at least at the base of the personalizable accessory, wherein the adhesive force between the load-bearing structural adhesive of the skin-adhered dermal adhesive and the skin surface exceeds the magnetic force generated between the load-bearing dermal adhesive and the magnetic accessory, wherein the magnetic force between the load-bearing dermal adhesive and the magnetic accessory exceeds the gravitational force acting on the magnetic accessory, and thereby, the interaction between the magnetic particles (incorporated into the load-bearing magnetic dermal adhesive) and the magnetic elements (incorporated into the magnetic accessory) stably attaches the magnetic accessory to a skin surface of interest.

In other embodiments, various load-bearing magnetic dermal adhesives capable of magnetically attaching a range of “magnetic personalizable accessories” to various skin surfaces of the face and body for aesthetic/stylish enhancement and/or entertainment/amusement are provided. The load-bearing magnetic adhesives comprise: (a) one or more magnetic particles suitable for use in a cosmetic formulation; and (b) a load-bearing structural adhesive, capable of adhering to a dermal surface and capable of magnetically supporting the attachment of a magnetic dermal accessory, wherein the cosmetic formulation is convertible from a non-bearing-load state to a load-bearing adhesive state after coating the skin surface; and wherein the adhesive force between the load-bearing structural adhesive of the skin-adhered “magnetic dermal adhesive” and the dermal surface is greater than the magnetic force between the load-bearing “magnetic dermal adhesive” and the “magnetic personalizable accessory,” so that the magnetic accessory is reversibly attachable and detachable.

In another embodiment, a magnetically attachable personalizable accessory is provided, comprising: (a) at least one magnetic accessory base incorporating one or more magnetic elements exhibiting high magnetic coercivity; and (b) a visible accessory surface of interest, wherein the magnetic elements are incorporated within the magnetic surface, and arranged to magnetically interact with the surface of a magnetic dermal adhesive film formable over any skin of interest.

In other embodiments, various methods for producing and using the various magnetically attachable magnetic personalizable accessories and the various load-bearing magnetic dermal adhesives are provided throughout the specification, including Examples 1-5 providing exemplary formulations for forming the load-bearing magnetic dermal adhesives of the present invention.

### BRIEF DESCRIPTION OF THE DRAWINGS

FIG. 1 illustrates a simplified hypothetical model of a magnetically attachable personalizable accessory system from a side perspective, showing the spatial alignment between a load-bearing magnetic dermal adhesive and a magnetically attachable personalizable accessory, as one embodiment.

FIG. 2A illustrates the hypothetical topical application of a load-bearing magnetic dermal adhesives to a skin of interest, as another embodiment.

FIG. 2B illustrates the hypothetical attachment of magnetically attachable personalizable accessories of interest, as another embodiment.

FIGS. 3A-D illustrate the hypothetical sequential attachment of magnetically attachable personalizable accessories (for decorating the forehead region) to the pre-coated, load-bearing magnetic dermal adhesives, as another embodiment.

FIG. 3E illustrate the hypothetical attachment of magnetically attachable personalizable accessories (for decorating noses and lips), as another embodiment.

FIG. 4A illustrates the hypothetical attachment of magnetically attachable personalizable accessories (for decorating necks via a partial choker design), as another embodiment.

FIG. 4B illustrates the hypothetical attachment of magnetically attachable personalizable accessories (for decorating necks via an artful necklace design), as another embodiment.

FIG. 5 illustrates the hypothetical attachment of magnetically personalizable accessories (for decorating shoulders), as another embodiment.

FIG. 6A-B illustrates the hypothetical attachment of multiple magnetically personalizable accessories of interest, over a pre-existing/pre-formed image, as another embodiment.

FIG. 7A illustrates the hypothetical attachment of two magnetically personalizable accessories that can be superimposed (for decorating feet as a two-dimensional design), as another embodiment.

FIG. 7B illustrates the hypothetical attachment of non-flat magnetically personalizable accessories (for decorating feet as a three-dimensional design), as another embodiment.

FIG. 8A illustrates the hypothetical spatial distribution of magnetic elements of the magnetic accessory base (a first configuration) for the magnetic personalizable accessories as another embodiment.

FIG. 8B illustrates the hypothetical spatial distribution of magnetic elements of the magnetic accessory base (a second configuration) for the magnetic personalizable accessories as another embodiment.

FIG. 8C illustrates the hypothetical spatial distribution of magnetic elements of the magnetic accessory base (a third configuration) for the magnetic personalizable accessories as another embodiment.

### DETAILED DESCRIPTION

### A. Definitions

The term “a” refers to one or more of an item/material of interest as typically referenced in patent documents.

The term “plurality” refers to one or more of an item/material of interest, interchangeable with the meaning of “many” or “several” or “a set.”

The terms “cosmetic” and “aesthetic” can be used interchangeably herein, and refer to the external/topical application of certain formulations or procedures intended to improve, enhance, decorate and/or restore a user's appearance, style, or beauty, including the magnetic attachment of one or more personalizable accessories for cosmetic/fashionable/costume enhancements, limited only by human imagination.

The term “pigment” refers to a coloring agent that can be derived from natural or synthetic sources in order to make the product more desirable from a user's perspective. The pigments suitable for making the load-bearing magnetic dermal adhesives can be selected from a range of materials providing the colors of interest, including black, brown, gray, blue, violet, red, green, orange, yellow and various blends thereof. As dermal adhesives, it is preferable to manufacture these formulations comprising pigment(s) that can visually match a broad range of naturally occurring skin tones.

The term “dermal compatible” refers to the biocompatibility relative to skin tissue for ensuring the safety of product users for the load-bearing magnetic dermal adhesives and the magnetic personalizable accessories.

The term “structural adhesives” refers to an adhesive capable of “hardening” or “curing” into a material capable of holding two or more substrates together in order to bear the forces suitable for the lifetime performance of the product.

The term “incorporating” refers to the inclusion or the addition of a material substance of interest in any manner, not limited to any known or unknown processes.

The terms “load-bearing magnetic dermal adhesive(s)” or “magnetic dermal adhesive(s)” of the present invention refer to various formulations incorporating one or more “magnetic particles” exhibiting strong ferromagnetic properties, and capable of supporting the physical attachment of one or more magnetic personalizable accessories based on magnetic forces of attraction.

Examples of dermal adhesive formulations include forms such as creams, ointments, pastes, paints or more dilute solutions commonly formulated for cosmetics and dermal compatible adhesives, without any specific “form” limitations.

The term “magnetic particles” (incorporated into the magnetic dermal adhesives) refers to materials preferably selected from suitable magnetic compounds, including without limitation iron oxides, such as hematite Fe₂O₃, Magnetite Fe^{II}(Fe^{III})₂O₄, super-paramagnetic iron oxides (SPIO), chromium dioxide CrO₂, barium ferrites (BaFe₁₂O₁₉, Ba₂ZnFe₁₈O₂₃, BaFe²⁺₂Fe³⁺₁₆O₂₇), Bismuth ferrite (BiFeO₃), Manganese-zinc ferrite (MnZn), Cobalt ferrite, CoFe₂O₄, Strontium ferrite, SrFe₁₂O₁₉, or yttrium iron granate (YIG) Y₃Fe₅O₁₂.

The terms “magnetic personalizable accessory” or “magnetic personalizable accessories” of interest refer to a broad range of accessories that can be formed by incorporating “magnetic elements” into the “magnetic accessory base,” and attachable to various skin surfaces of the face and body for aesthetic/fashionable enhancement and/or entertainment/amusement. The term “magnetically attachable personalizable accessory” can be used interchangeably with “magnetic personalizable accessory” in either singular or plural forms throughout this disclosure as embodiments of the present invention.

The term “magnetic accessory base” refers to the structural component of the “magnetically attachable personalizable accessory” that can incorporate one or more “magnetic elements” positioned and oriented along the magnetic accessory base, exhibiting high coercivity in the presence of the “load-bearing magnetic dermal adhesive” of the present invention.

The term “magnetic elements” (incorporated into the “magnetic accessory base”) refers to permanent magnets of the hard or soft type, such as iron, cobalt, nickel; alloys, such as samarium-cobalt alloys, including SmCo₅, Sm₂Co17, optionally alloyed with Fe, Cu, and Zr; neodymium-iron-boron Nd₂Fe₁₄B optionally alloyed with Pr and Dy, commonly referenced as “rare-earth” or “super magnets”; and/or other alloys such as Bismanol and AlNiCo.

The term “magnetic” and “ferromagnetic” are used interchangeably in this disclosure without any limitations.

The terms “attachable” or “adherable” are used interchangeably in reference to the ability to physically interact with another surface of interest resulting in the bonding of two contacting surfaces, wherein the bonding is reversible as used in relation to the interaction between the “load-bearing magnetic dermal adhesives” coated over a skin of interest and one or more “magnetic personalizable accessories,” and in relation to the interaction between the “magnetic accessory base” and the skin adhered/pre-coated “magnetic dermal adhesive.”

The term “skin of interest” refers to any dermal tissue located on a human being, including any skin forming the facial features such as the forehead, above and below the eyes, the cheeks, the noses, the ears, the lips, and surrounding regions. Skin of interest also includes any skin forming the “body,” such as the necks, the shoulders, the chests, the upper/lower backs, the abdomens, the arms, the legs, the hands, and feet.

The term “non-load-bearing” state refers to a state in which a load cannot be supported by an adhesive.

### B. The Magnetically Attachable Personalizable Accessory System

Product consumers are increasingly interested in wearing personalizable products that can be an enjoyable form of self-expression to enhance the quality of one's experience, similar to the use of personalizable jewelry, tattoos, dermal stickers, and other cosmetic/fashionable adornments designed to make direct contact with consumers' skin.

For example, women consumers especially appreciate wearing cosmetic/fashionable personalizable products that can be intelligently designed, lighter in weight, aesthetically pleasing, comfortable and convenient to use (i.e., “user friendly” in meeting customer expectations). For special personal and professional occasions/events, such as live/recorded performances, holiday festivities, celebrations, and wedding ceremonies, many women enjoy wearing a broad variety of faux gems, sparkly sequins, and other glamour-enhancing accessories for their faces and bodies to enhance their attractiveness/visibility. However, these glamour-enhancing accessories can be typically attachable via sticker-like, weak or temporary adhesives, meaning that they cannot be re-useable because the adhesive portion can be “less sticky” upon, interaction with greasy/sweaty human skin, preventing effective re-uses and are discarded after a single use, generally deemed environmentally wasteful and socially irresponsible.

Furthermore, experienced product developers appreciate that any products designed for children, such as decorative or costume stickers applicable over their sensitive skins, are ideally formulated to avoid undue irritations that can be activated by many environmental/chemical agents. These “sticker-attachable” costume accessories are popular among children and adults because they enhance any holiday costumes, role-playing outfits, and various “play suits” suitable for many children and adult social situations/events.

Recently, a broad range of temporary stickers resembling tattoos are gaining massive popularity because of their temporary nature, unlike permanent tattoos, which can be painful and extremely costly to professionally remove using laser technology, often resulting in large regions of visible scars and emotional trauma. Consumers enjoy embellishing their skin with various cosmetic makeup, body paint, tattoos, tattoo-like stickers, costume stickers, piercing and non-piercing jewelry, and other costume/fashionable accessories for various occasions and personal/professional uses.

The present invention provides a convenient way for reversibly attaching/detaching/interchanging various magnetic personalizable accessories to interact with consumers' skin so that they can be (a) “re-useable” saving money, time, and resources; (b) can be arranged in various ways (worn singly or grouped together in an infinite number of unique arrangements); (c) can provide a broad range of options for creative self-expression/personal style; (d) can be interchangeably attached to achieve multiple variations; (e) can be conveniently swapped/exchanged from one user to another (f) can be stacked or superimposed together into various creative arrangements; (g) can be utilized for enhancing the experience of various personal and professional situations; and many more benefits that can be uniquely crafted by each consumer according to his/her particular performance requirements.

The inventive concept is directed to a magnetically attachable personalizable accessory system comprising two main components: a load-bearing magnetic dermal adhesive and a magnetically attachable personalizable accessory comprising a “magnetic accessory base.” These two magnetic complementary products are designed to magnetically attract each other during intended product use because the magnetic dermal adhesive incorporates “magnetic particles” suitable for cosmetic/dermal topical applications, wherein the “magnetic particles” can magnetically attract the “magnetic accessory base” of personalizable accessories, and thereby, supporting the stable attachment of the various personalizable accessories of interest.

In several embodiments, the present invention is directed to a load-bearing magnetic dermal adhesive capable of attaching one or more magnetic personalizable accessories, comprising: (a) a pigment suitable for cosmetic formulation; (b) one or more magnetic particles suitable for use in a pigmented cosmetic formulation; and (c) a load-bearing structural adhesive, capable of adhering to the surface of a skin of interest and capable of magnetically supporting the attachment of a magnetic personalizable accessory, wherein the cosmetic formulation is convertible from a non-load-bearing state to a load-bearing adhesive state after coating the skin surface; and wherein the adhesive force between the load-bearing structural adhesive of the skin-adhered dermal adhesive and the skin surface is greater than the magnetic force between the load-bearing dermal adhesive and the personalizable accessory, so that the personalizable accessory is reversibly attachable and detachable.

In several embodiments, the present invention is directed to a magnetically attachable personalizable accessory, comprising: (a) an accessory base incorporating one or more magnetic elements exhibiting high magnetic coercivity; and (b) a visible accessory surface of interest, wherein the magnetic elements are positioned in a portion of the accessory base, and spatially arranged/distributed to maximize the interaction with the surface of a magnetic dermal adhesive film formable over a skin of interest.

In several embodiments, various methods for producing and using the various magnetically attachable personalizable accessories and the various load-bearing magnetic dermal adhesives are provided throughout the specification. Furthermore, Examples 1-5 provides exemplary formulations for forming the load-bearing magnetic dermal adhesives of the present invention.

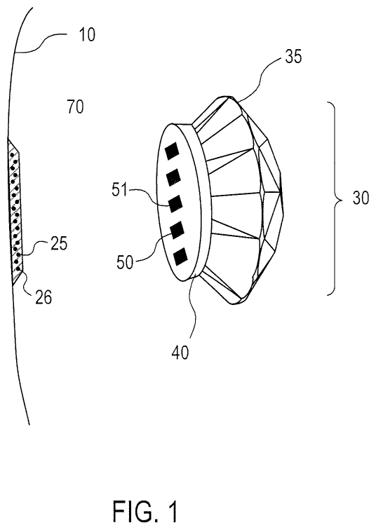

The magnetic interaction between the main components of the magnetically attachable personalizable accessory system can be explained in FIGS. 1-7 below.

FIG. 1 illustrates a simplified hypothetical model of a magnetically attachable personalizable accessory system from a side perspective, showing the spatial alignment between a load-bearing magnetic dermal adhesive and a magnetically attachable personalizable accessory, as one embodiment. In FIG. 1, a hypothetical magnetic dermal adhesive 70 comprising magnetic particles 25 and 26 can be topically applied over a skin of interest 10 as shown. In spatial alignment with respect to the pre-coated magnetic dermal adhesive film, a magnetically attachable personalizable accessory of interest 30 is shown, comprising a visible accessory surface of interest (e.g., gem-like, crystal-cut appearance as shown) and a magnetic accessory base 40 incorporating one or more magnetic elements 50 and 51. Thus, the magnetically attachable personalizable accessory system comprises a load-bearing “magnetic dermal adhesive” and a “magnetic personalizable accessory,” as shown. Exemplary magnetic dermal adhesive formulations comprising various ferromagnetic particles are provided in Examples 1-5. The magnetic personalizable accessories can be attached/detached reversibly so that they can be re-usable and interchangeable with different types of such accessories.

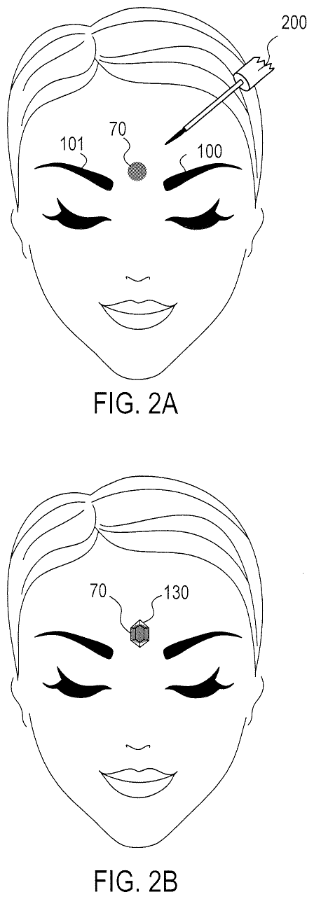

FIG. 2A illustrates the hypothetical topical application of a load-bearing magnetic dermal adhesives to a skin of interest, as another embodiment. In FIG. 2A, a magnetic dermal adhesive can be topically applied to a woman's forehead region between the eyebrows 100 and 101 as shown, to form a magnetic dermal adhesive film that can function as an anchor for the attachment of various magnetic personalizable accessories of interest. The a magnetic dermal adhesive film can be formed on any skin, providing substantial flexibility in where the magnetic personalizable accessories can be attached, ranging from relatively stable surfaces (such as the chest area) to dynamic surfaces such as one's neck, which experiences a broad range of motion.

FIG. 2B illustrates the hypothetical attachment of magnetically attachable personalizable accessories of interest, as another embodiment. In FIG. 2B, a faux gem piece 130 can be superimposed over a pre-coated film comprising the magnetic dermal adhesive film 70. It is desirable to match the size of the dermal adhesive film (average diameter) to the size of the personalizable accessories (average diameter) so that the dermal adhesive film cannot be visible from multiple perspectives (as shown). When the magnetic forces of attraction between the magnetic dermal adhesive film 70 and the magnetic accessory 130 can be sufficiently strong, the personalizable accessories can be firmly adhered so that typical facial motions and/or body movements would not de-stabilize their skin adherence.

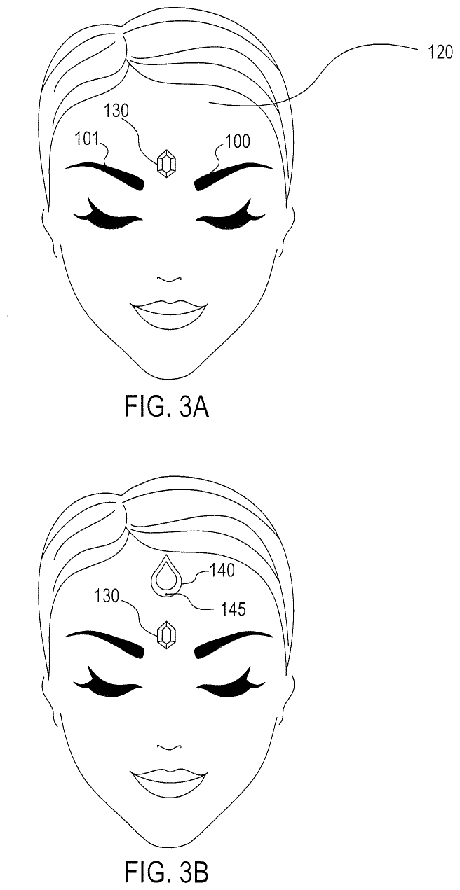

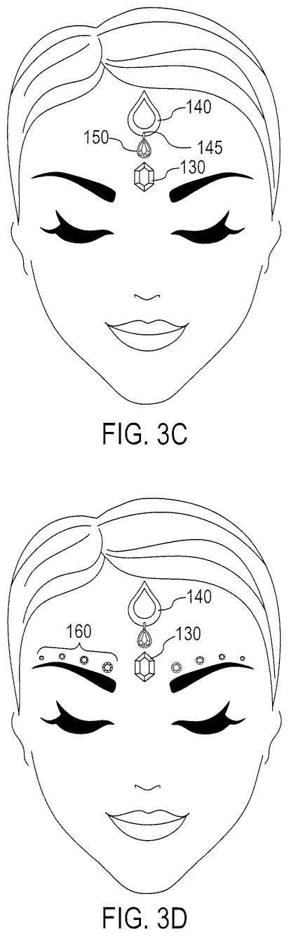

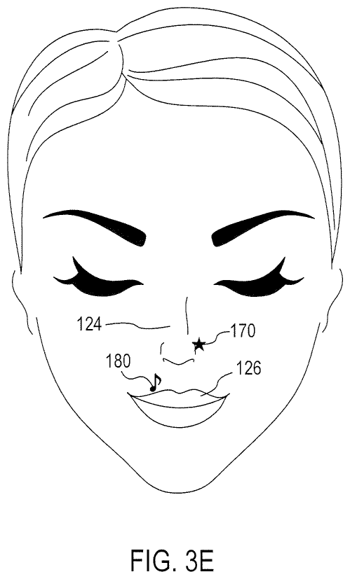

FIGS. 3A-D illustrate the hypothetical sequential attachment of magnetically attachable personalizable accessories (for decorating the forehead region) to the pre-coated, load-bearing magnetic dermal adhesives, as another embodiment. In FIG. 3A, a first gem piece 130 can be positioned and magnetically attached to the forehead region 120 and between eyebrows 100 and 101 as shown. In FIG. 3B, a second gem piece 140 can be positioned and magnetically attached to the forehead above the first gem piece 130. In this example, the second gem piece 140 includes a hole 145 to optionally insert a third gem piece if desired. In FIG. 3C, a third gem piece 150 can be attached to gem 140 by threading a hook/ring through the hole 145 as shown. The use of a hook/ring provides substantial range of movement and interchangeability if desired. In FIG. 3D, four gem pieces can be attached over the left brow 160 and over the right brow (shown without reference numbers). Thus, FIGS. 3A-D illustrate various arrangements that can be possible by magnetically attaching the personalizable accessories of interest, limited only by the human imagination.

FIG. 3E illustrate the hypothetical attachment of magnetically attachable personalizable accessories (for decorating noses and lips), as another embodiment. In FIG. 3E, a star-shaped magnetic personalizable accessory 170 is shown magnetically attached to a user's nose 124. A magnetic personalizable accessory shaped as a musical note 180 can be magnetically attached to the lips 126 as shown. These examples represent the “faux piercing” concept, in which noses/lips can be adorned with attractive personalizable accessories without undergoing painful piercing experiences and/or infection susceptibilities. Other skin types may be modified in a similar manner to appear as “pierced,” avoiding such discomfort/infection.

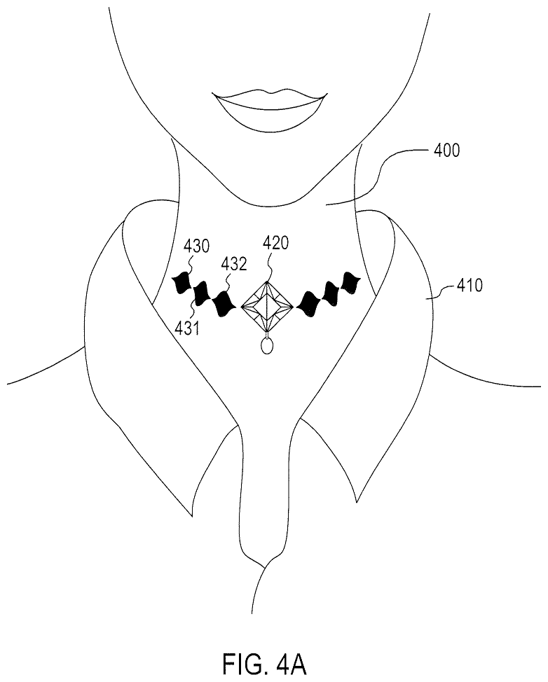

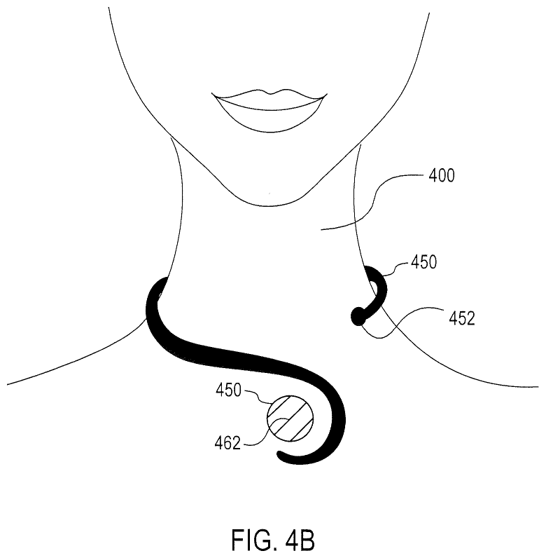

FIG. 4A illustrates the hypothetical attachment of magnetically attachable personalizable accessories (for decorating necks via a partial choker design), as another embodiment. In FIG. 4A, a hypothetical choker design is shown, in which the various gem-like pieces of personalizable accessories are spatially arranged in a particular sequence and positioned along a woman's neck 400, resulting in an attractive presentation comprising a center piece 420 surrounded by the symmetrical distribution of smaller “bead-shaped” personalizable accessories 430, 431, and 432 on both sides. This can be especially suitable if one does not enjoy feeling a bulky choker/necklace when wearing a high collar shirt 410, as shown.

FIG. 4B illustrates the hypothetical attachment of magnetically attachable personalizable accessories (for decorating necks via an artful necklace design), as another embodiment. In FIG. 4B, a two-piece magnetic personalizable necklace is shown, comprising a choker 450 that can be artistically wrapped around a woman's neck 400, comprising at least one magnetic anchor point 452 (for extra stabilization) and a second accessory piece 450 that can be positioned and separately attached to a woman's skin in another area, in a manner that the overall presentation can be visually pleasing. The visible accessory surface of interest 462 can be modified in multiple variations (and interchangeable) to suit the personal preferences of consumers. The magnetic anchor point 452 and the second accessory piece 450, respectively, can be attached to the skin by topically pre-coating the skin area of interest with the magnetic dermal adhesive (not shown).

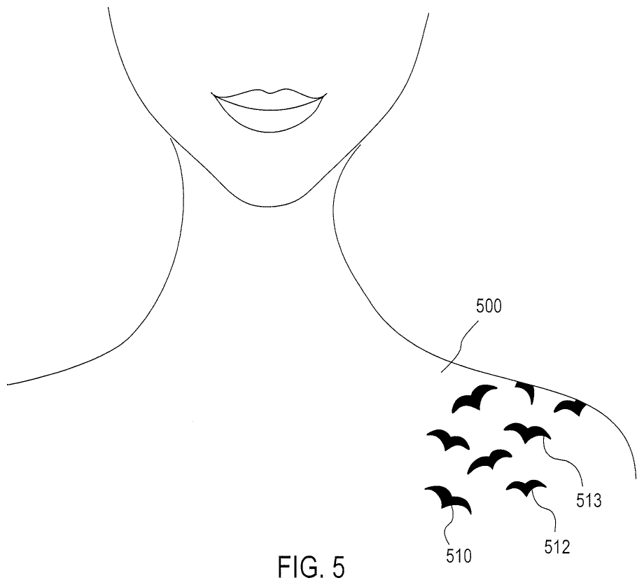

FIG. 5 illustrates the hypothetical attachment of magnetically personalizable accessories (for decorating shoulders), as another embodiment. In FIG. 5, multiple numbers of magnetic accessories shaped as birds 510, 512, and 513 can be spatially arranged along a woman's shoulder 500 in various ways to create a visually pleasing arrangement. Grouping of multiple magnetic accessories can be applied to any region of the face or body, producing visually dramatic effects as desired, limited only by the human imagination.

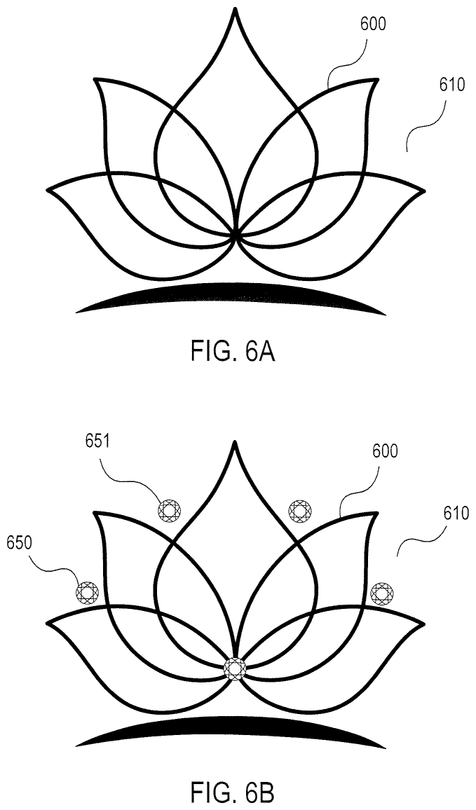

FIG. 6A-B illustrates the hypothetical attachment of multiple magnetically personalizable accessories of interest, over a pre-existing/pre-formed image as another embodiment. In FIG. 6A, a lotus flower tattoo design (temporary or permanent) 600 can be placed over a skin region 610 of interest as shown. In FIG. 6B, five exemplary magnetic personalizable accessories (such as 650 and 651) can be magnetically attached over a pre-existing tattoo for embellishing a lotus flower tattoo design, as shown. The drawing or image can be formed manually by painting, stamping, printing, and/or utilizing any pigment transferring techniques. Any type of body art/body piercing can be embellished in this manner.

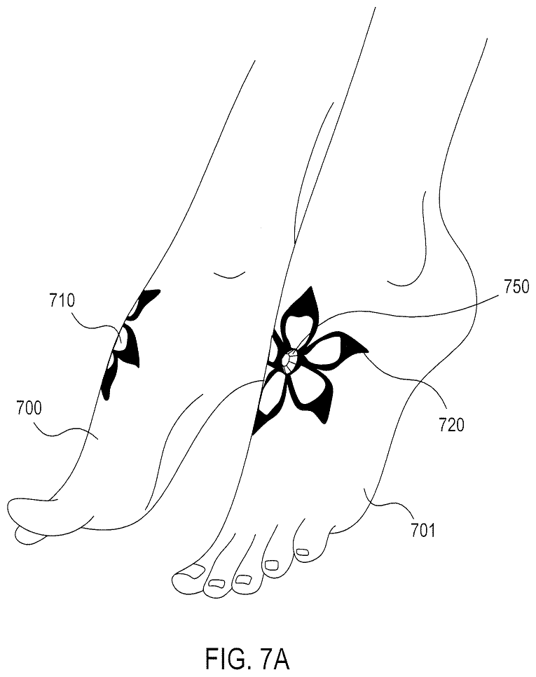

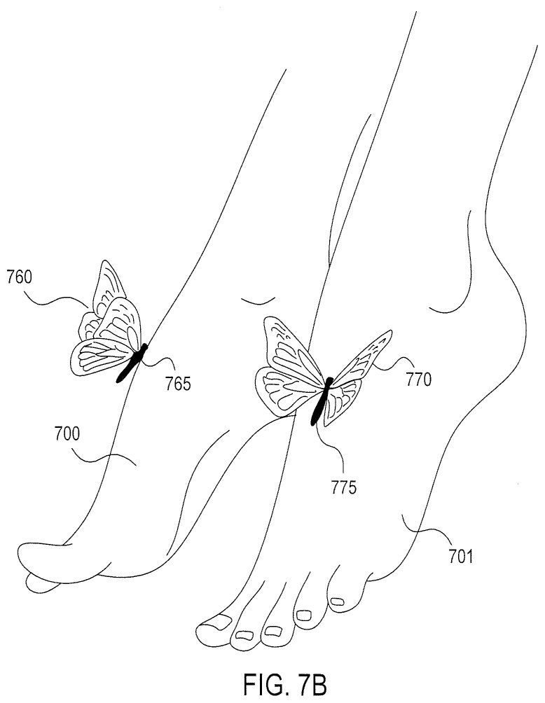

FIG. 7A illustrates the hypothetical attachment of two magnetically personalizable accessories that can be superimposed (for decorating feet as a two-dimensional design), as another embodiment. In FIG. 7A, the first magnetic accessory represents a faux gem 750 attached over a second magnetic accessory (shaped as a flat flower) 720 (or 710 on foot 700) as shown, wherein both magnetic accessories can be magnetically attached to feet 700 and 701.

FIG. 7B illustrates the hypothetical attachment of non-flat magnetically personalizable accessories (for decorating feet as a three-dimensional design), as another embodiment. In FIG. 7B, the first magnetic accessory (a first butterfly) 760 comprising a magnetic accessory base 765 and the second magnetic accessory (a second butterfly) 770 comprising a magnetic accessory base 775 can be magnetically attached to feet 700 and 701, respectively as shown. This embodiment represents various situations in which the magnetic personalization accessories can be non-flat, exhibiting 3D qualities, such as faux mustaches for costumes, costume eye masks, costume bows, costume ribbons, among many accessory variations that may be conceivable for a broad range of performance situations and social events.

### C. Functional Components of Load-Bearing Magnetic Dermal Adhesive Formulations

### Exemplary Structural Adhesives

In general, adhesives can be defined as substances capable of holding at least two surfaces together. A wide range of adhesive formulations are conceivable, and can be differentiated into either “pressure-sensitive adhesives” (PSAs) and “structural adhesives.” Pressure-sensitive adhesives (PSAs) can adhere strongly to solid surfaces upon application of light contact pressure and short contact duration, useful for manufacturing “post it” notes, adhesive pads, pressure-sensitive tapes, various labels, and a wide variety of products. Three different types of PSAs commercially employed are described as either: a) organic solvent based, b) water-based (emulsion), or c) thermoplastic. Pressure-sensitive adhesives are appreciated for their ease of application and removal from surfaces, but exhibit relatively less load-bearing capability than structural adhesives, and can be more prone to inadvertent detachment from substantially moist/oily surfaces, similar to most skin surfaces. Alternatively, structural adhesives can be defined as adhesives that “harden” or “cure” into a material capable of stably holding two or more substrates together during the expected lifetime of the product. Structural adhesives are often termed “load-bearing” adhesives, and exhibit higher load-bearing capabilities than aforementioned PSAs. Structural adhesives can be formulated utilizing various polymer chemistries, including polyurethane-, acrylic-, silicone-, vinyl-, or epoxy-based polymers and blends, among other suitable materials known by persons skilled in the art.

With respect to skin-contacting adhesive formulations, a variety of different types of adhesives can be utilized to enable skin-bonding. Suitable examples include various dermal adhesives intended to support wound closure/wound repair. These formulations utilize among others, cyanoacrylate monomers and monomer blends formed from 2-octyl cyanoacrylate (CAS 133978-15-1) and/or n-Butylcyanoacrylate. Other dermal formulations may include Octyl 2-cyanoacrylate (CAS 6701-17-3), Isobutyl 2-cyanoacrylate and various materials that are functional equivalents.

In selecting suitable skin-contacting adhesives, cyanoacrylate formulations may be limited for use as a dermal adhesive in that they can be polymerized in the presence of water, and can polymerize rapidly (from tens of seconds to few minutes) in a manner that can be challenging to control for skin applications. These formulations must be stored dry to prevent their activation, since they can initiate curing upon contact with a moist skin. Thus, they must be applied comparatively quickly, and spread accurately. Because their adhesion to skin is quite substantial, and can last for a comparatively long time period (approximately one to several days), such formulations may not be suitable for formulating dermal adhesive products, in that: a) these formulations will not permit a sufficiently intermittent cosmetic application during 4-12 hour period, and b) the premature removal from a skin surface may require substantial force to be exerted and result in substantial discomfort. Cured formulations are also difficult to remove by traditional detergents, including soap and water, so that more volatile and potentially hazardous solvents, such as ethanol, ethyl acetate, or acetone may be needed to remove films/layers formed on the skin from such formulations. However, these types of solvents are known to degrease the skin and may cause skin irritations. Because the skin of some consumers can be very sensitive, it is desirable to avoid using such volatile, irritating and potentially hazardous solvents during dermal adhesive product use/product removal. Thus, there is a specific need to find more suitable skin-adhering formulations that do not detach as easily from the skin as aforementioned PSAs, and that does not adhere as strongly, as in the example of the aforementioned cyanoacrylate-based structural adhesives.

### Exemplary Pigments

The addition of pigments enables the magnetic dermal adhesives to be formed in any desirable color from a spectrum of hues. Suitable pigments can be chosen from natural or synthetic, metallic, nonmetallic, inorganic pigments, organic pigments and/or pearlescent pigments, depending on the desired visual appearance, flowability, spreadability, dispersability, and/or wetting ability. Suitable pigments can be provided a) in various particle size ranges from typically tens of nanometers to about several hundred micrometers; b) with or without surface treatments; c) in hydrated or dehydrated form; and d) as is, or blended with each other, to reach a desired color spectrum. Exemplary inorganic pigments include black, yellow, red and brown iron oxides; titanium dioxide (rutile or anatase) (white), manganese violet; ultramarine blue; chromium oxide (green) and ferric blue, among others. Organic pigments include pigments D & C yellow, D & C orange, D & C red, carbon black and other dyes and/or lakes, among others. Pearlescent pigments include white pearlescent pigments, such as mica coated with titanium oxide or bismuth oxychloride; colored pearlescent pigments such as titanium mica with iron oxides, titanium mica with ferric blue or chromium oxide, titanium mica with an organic pigment of the abovementioned type, and pigments based on bismuth oxychloride, among others.

### Exemplary Magnetic Particles and Magnetic Elements

For producing the magnetically attachable personalizable accessories, it is desirable to select (a) suitable “magnetic particles” exhibiting strong ferromagnetic properties for incorporation within the magnetic dermal adhesive formulations; and (b) suitable “magnetic elements” exhibiting strong ferromagnetic properties for incorporation within the “magnetic accessory base.” These magnetic elements can be employed as: (i) a filler agent, as (ii) a color-imparting component, and (iii) a magnetic material that can facilitate the anchoring/attachment of the “magnetic accessory base” to the present “load-bearing magnetic dermal adhesive.”

The ferromagnetic properties of the magnetic particles and the magnetic elements must be compatible in order for the magnetic accessory base to attach stably to the magnetic dermal adhesive (skin-adhered/pre-coated state). As a preferred embodiment, a ferromagnetic material exhibiting high coercivity is suitable for use in forming “magnetic particles” incorporated within the “load-bearing magnetic dermal adhesive” formulation, preferably in the range between 10-25,000 kA/m, more preferably from 100-15,000 kA/m and most preferably from 1,000-10,000 kA/m. As a preferred embodiment, a “magnetic element,” having an intrinsically high coercivity is incorporated into the “magnetic accessory base,” preferably in the range between 10-25,000 kA/m, more preferably from 100-15,000 kA/m and most preferably from 1,000-10,000 kA/m.

As another embodiment, suitable materials for forming the “magnetic elements” for incorporation into the “magnetic accessory base” include powderized/micronized ferromagnetic elements, ferromagnetic alloys and ferromagnetic compounds, typically used in permanent magnets of the hard or soft type, such as iron, cobalt, nickel; alloys, such as samarium-cobalt alloys, including SmCo₅, Sm₂Co17, optionally alloyed with Fe, Cu, and Zr; neodymium-iron-boron Nd₂Fe₁₄B optionally alloyed with Pr and Dy, commonly referenced as “rare-earth” or “super magnets”; and/or other alloys such as Bismanol and AlNiCo.

As another embodiment, suitable materials for forming “magnetic particles” for incorporation into the “magnetic dermal adhesives” include ferromagnetic compounds such as oxide-based magnetic pigments, including without limitation iron oxides, such as hematite Fe₂O₃, Magnetite Fe^{II}(Fe^{III})₂O₄, super-paramagnetic iron oxides (SPIO), chromium dioxide CrO₂, barium ferrites (BaFe₁₂O₁₉, Ba₂ZnFe₁₈O₂₃, BaFe²⁺₂Fe³⁺₁₆O₂₇), Bismuth ferrite (BiFeO₃), Manganese-zinc ferrite (MnZn), Cobalt ferrite, CoFe₂O₄, Strontium ferrite, SrFe₁₂O₁₉, or yttrium iron granate (YIG) Y₃Fe₅O₁₂.

As another embodiment, the “magnetic elements” and “magnetic particles” can be provided in encapsulated or unencapsulated form to optimize properties such as suspendability, dispersability, surface wetting characteristics, dermal compatibility, and resistance from environmental factors, including oxidation resistance. For example, iron oxides can be prevented from environmental degradation or direct skin contact by the addition of masking agents, including fatty acids, such as undecylenic, lauric or oleylic acid.

With regard to suitable “magnetic particle” sizes for use in the magnetic dermal adhesive formulation, generally, the preferred size ranges cannot easily be visually distinguished from one another by the naked eye, thus avoiding a granular appearance. As several embodiments, the magnetic particles exhibit an average diameter size ranging from 1 μm to 1 mm, preferably ranging from 5 μm to 500 μm, and most preferably ranging from 50 μm to 250 μm. As another embodiment, the magnetic particles exhibit an average diameter size ranging from 100 nm to 1 μm, wherein the magnetic particles can be further encapsulated to inhibit sub-dermal transport.

Black iron oxides, which are available in various size ranges and chemistries, are deemed particularly suitable, because they impart not only a rich black color, but also confer the desired magnetic properties to the underlying formulation. A suitable overall composition range for such iron oxide based magnetic particles can range from between 10-60 (w/w %), more preferably 20-50 (w/w %), and most preferably from 25-45 (w/w %). It should be noted, that the higher the coercivity of the “magnetic particle” chosen as the pigment component, the lesser amount need be incorporated to achieve a desired magnetic adhesion force for stably attaching a “magnetic accessory base” to a complementarily formulated “magnetic dermal adhesive.” An analogous rationale can be applied with respect to the incorporation of magnetic elements into the “magnetic accessory base.” Thus, the needed magnetic adherent force can be finely controlled through material selection and the relative amount of “magnetic elements” added to the “magnetic accessory base” with respect to the amount of “magnetic particles” added to the “magnetic accessory base.”

### Suitable Adhesive and Viscosifying Components

The inventors of the present application have found that suitable skin-adhering formulations can be formed on the basis of acrylate polymer blends, preferably from methyl and ethyl acrylate, 2-hydroxyethylacrylate, butyl acrylate, isobutyl acrylate, t-butyl methacrylate, isooctyl acrylate, ethyl hexyl acrylate, isobornyl acrylate, vinyl acetate, and more particular from: ethyl hexyl acrylate copolymers in an overall weight ratio of 0-30 (w/w %), more preferably 5-25 (w/w %) and most preferably from 10-15 (w/w %) in combination with butyl acrylate, methyl methacrylate, and methacrylic acid copolymers, in an overall weight ratio of 5-45 (w/w %), more preferably 10-30 (w/w %) and most preferably from 15-25 (w/w %), that can be selected from a molecular weight range of 5,000-30,000 g/mol, more preferably from 10,000-20,000 g/mol and most preferably from 15,000-20,000 g/mol. The latter polymer blends can be particularly suitable for forming a structural adhesive capable of stably adhering to a skin of interest.

### Suitable Thickening Agents

Depending on the desired flowability and spreadability for an optimized application to the skin, viscosifying-, gelling-, thickening-, bulking- and/or thixotropic agents can be added to facilitate the blending with other acylates in the liquid formulations. Examples include gelling agents based on natural gums, including carob gum, guar gum, gum arabic, karaya gum, gum tragacanth and ghatti gum, agar-agar, carrageenans, alginates, gelatin, caseinates, albumins, pectins, starches, polysaccharides, such as xanthan gum, chitin and/or chitosanes, polydextranes such as carboxymethyl dextranes, cellulose and cellulose derivatives, such as carboxymethylcellulose, hydroxypropylcellulose, methylcellulose, hydroxyl-propylmethylcellulose or hydroxyethylcellulose, and cellulose derivatives modified by alkyl- or alkoxy-groups. Others include inorganic filler materials, and/or pigments, such as silicate minerals, talcs or hydrated magnesium silicates, micas or aluminosilicates, such as for example muscovite, margarite, roscoelite, lipidolite, biotite, sericite, hectorite and/or bentonite, kaolin or hydrated aluminium silicate, boron nitride, fumed silica and/or titanium dioxide. Naturally occurring resins, fragrances, oils, and waxes of plant or animal derived materials among others can be added to further enhance the performance of the dermal adhesives.

### Suitable Film-Forming Components

In formulating the load-bearing magnetic dermal adhesives, other desirable properties include cosmetic functions such as film-forming and opacifying-controlling properties that can facilitate the formation of a homogenous, visually pleasing opaque film surface. It is noted that these properties can be enhanced through the addition of styrene/methacrylate/acrylate copolymer blends, including, but not limited to methacrylic acid-styrene copolymer and/or their respective alkali salts. Suitable overall weight ratios in a formulation comprising such styrene/acrylate copolymer blends can include 5-45 (w/w %), more preferably 10-30 (w/w %) and most preferably from 15-25 (w/w %). In the event, that more water resistant film-forming properties are desirable, fluorinated acrylate polymers, such as 2,2,2-Trifluoroethyl methacrylate, 2,2,3,3-Tetrafluoropropyl methacrylate, 2,2,3,4,4,4-Hexafluorobutyl methacrylate, 2,2,3,3,3-Pentafluoropropyl acrylate, and/or 1,1,1,3,3,3-Hexafluoroisopropyl acrylate can be added to the blend in a suitable proportion to weatherproof the surface.

### Suitable Water Content

Overall water weight ratios for the provided formulations can be chosen from between 0-50 (w/w %) more preferably from 0-25 (w/w %) and most preferably from 5-15 (w/w %). Upon application, the water evaporates up to a residual content (e.g. equivalent to the moisture retained in the skin), substantially solidifying the adhesive formulation in the process, and thereby creating a stable structural support and adherence on the skin substrate.

### Desired Viscosity Ranges

The viscosity can be adjusted through a desired amount of viscosifying agents, thickening agents, and/or filling agents, particularly including various pigments. The viscosity of the formulations in the underlying disclosure typically can range from 5 cps-300 cps, which can be measured between about the viscosity of water and castor oil (or higher viscosity).

The aforementioned polymer blends do not cure in the presence of water because they are not provided as a monomer, and are already cured. They can be readily dissolved in water, forming viscous to gel-like solutions and are suitably provided in a non-crosslinked state. Because the formulations can be formed partially from water-soluble, non-crosslinked polymers, the magnetic dermal adhesive film/layer can be easily removed from the skin by employing conventional detergent solutions and associated skin-care products intended for make-up or adhesive removal.

### Emollients/Humectants

To further control the residual moisture content and hardness of the adherent layer, various humectifying-, emollifying-, softening- and/or anti-caking agents can be added to the adhesive formulations. Suitable humectants can include ethylene-, propylene-, butylene- and hexylene glycols, as well as their multifunctional alcohol derivatives, alpha-hydroxy acids such as lactic acid, glyceryl triacetate, polymeric polyols such as polydextrose, sodium hexametaphosphate, sugar alcohols such as glycerol, sorbitol, xylitol, maltitol, and urea. Suitable emollients can include such as castor oil, cetyl alcohol, cetearyl alcohol, cocoa butter, isopropyl myristate, isopropyl palmitate, lanolin, liquid paraffin, polyethylene glycols, shea butter, silicone oils, stearic acid, and stearyl alcohol. Suitable humectifying-, emollifying-, softening- and/or anti-caking agents concentrations in the overall composition can range from between 0.01-20 (w/w %), more preferably 0.1-15 (w/w %), and most preferably from 1-10 (w/w %).

### Surfactants and Dispersants

To achieve a homogeneous dispersion of the substantially solid components of the formulation, the addition of surfactants and emulsifying agents such as saturated and unsaturated polyoxyethylene (2-80) alkyl (C8-20) ethers, for example Polysorbate 20-80, or polyethylene glycol ethers derived from castor oil, for example Ricinoleth-40 can be added.

Several exemplary formulations for making the load-bearing magnetic dermal adhesives are further described in Examples 1-5. Alternative substitutions that are conceivable by persons skilled in the art of cosmetic formulations are contemplated within the scope of the present invention.

### D. Various Configurations for Arranging the Magnetic Elements of the Magnetic Accessory Base to Enhance Magnetic Attraction Forces

Persons skilled in the art of manufacturing various magnetic products appreciate that the relative orientation, distance and structural arrangement of one or more magnetic particles/elements in a spatial relationship to each other can directly impact the resulting magnetic field strength/flux density formed around/within the combined body of such magnetic particles/elements. The application of a series of geometric distributions or arrays of magnetic elements into product designs can be desirable in situations where preferential enhancement of a magnetic field can be preferred along one or more particular surfaces of a product. One such specific configuration of magnets is referenced as a Halbach array, known to augment or strengthen the magnetic field along one side of the array while cancelling the magnetic field along the other side. By employing Halbach arrays into the magnetic product designs, manufacturers can potentially use less material to make a functioning magnet capable of exhibiting a stronger magnetic field along one preferential surface and simultaneously ensuring that the other opposing surface can be magnetically weaker to serve a non-magnetic function, such as providing a surface for attaching/interacting/gluing other materials or components, or to prevent/minimize such surface from magnetically adhering to another ferromagnetic surface.

For example, a refrigerator magnet can be manufactured by co-extruding powdered ferrite (iron rust) and polymeric binding agents. When the magnet is exposed to a rotating magnetic field, a one-sided flux distribution, analogous to the described Halbach array can be created. This distribution can increase the holding force of the magnet when placed on a permeable surface, compared to the holding force that can be obtained from a uniform magnetization of the magnetic compound.

In a similar manner, the “magnetic elements” for incorporation within the “magnetic accessory base”, formable from a series of small magnetic strips, can be oriented in a deliberate pattern/orientation, so that the magnetic field can supplement each other along one preferential side of the accessory base, while preferentially cancelling each other along the back side (opposing side). In general, the magnetic products that can be derived by applying these one-sided flux distributions can exhibit stronger magnetic fields (i.e., up to about two times more magnetic force relative to an uniform magnetization) across one preferential surface, and can exhibit a substantially weaker field across the other (opposing) surface of a magnetic product of interest.

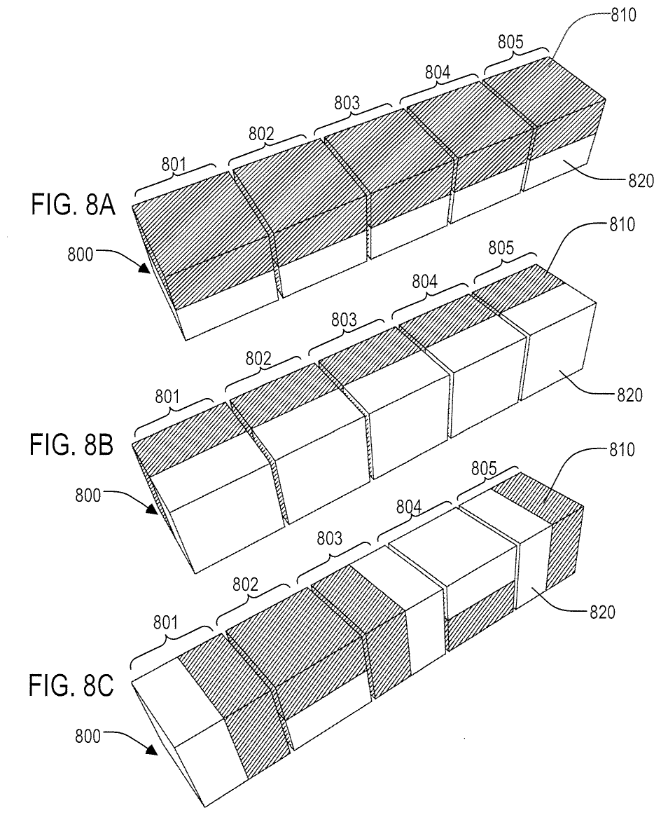

FIGS. 8A-C provide examples for controlling the spatial distribution of magnetic elements incorporated into the accessory base to augment/enhance the orientation and strength of the magnetic field for various purposes, as several embodiments illustrated below.

FIG. 8A illustrates the hypothetical spatial distribution of magnetic elements of the magnetic accessory base (a first configuration) for the magnetic personalizable accessories as another embodiment. In FIG. 8A, permanent magnetic elements 801-805 can be spatially arranged 800 viewing from left to right as shown. Each magnetic element comprises a “north pole” 810 and a “south pole” 820 indicating the polarity and/or orientation of the magnetic flux present in each element. In FIG. 8A, the series of permanent magnetic elements are spatially arranged so that the north poles are oriented perpendicular to and pointing towards the side that faces the pre-coated dermal adhesive film. In this manner, the spatial distribution of the magnetic elements of interest can be oriented during the manufacture of the magnetic accessory base that can exhibit enhanced magnetic fields/properties, thereby, enabling strong/stable attachment of various magnetic personalizable accessories directly onto the top surface of (i.e., perpendicular to) the pre-coated dermal adhesive film if desired.

FIG. 8B illustrates the hypothetical spatial distribution of magnetic elements of the magnetic accessory base (a second configuration) for the magnetic personalizable accessories as another embodiment. In FIG. 8B, permanent magnetic elements 801-805 can be spatially arranged 800 viewing from left to right as shown. Each magnetic element comprises a “north pole” 810 and a “south pole” 820 indicating the polarity and/or orientation of the magnetic flux present in each element. In FIG. 8B, the series of permanent magnetic elements can be spatially arranged so that the north poles are oriented in parallel/along the length of the (opposing) side facing the pre-coated dermal adhesive film. In this manner, the spatial distribution of the magnetic elements of interest can be oriented during the manufacture of the magnetic accessory base that can exhibit enhanced magnetic fields/properties, thereby, enabling strong/stable attachment of various magnetic personalizable accessories directly to a side surface of the pre-coated dermal adhesive film if desired.

FIG. 8C illustrates the hypothetical spatial distribution of magnetic elements of the magnetic accessory base (a third configuration) for the magnetic personalizable accessories as another embodiment. In FIG. 8C, permanent magnetic elements 801-805 can be spatially arranged 800 viewing from left to right as shown. Each magnetic element comprises a “north pole” 810 and a “south pole” 820 indicating the polarity and/or orientation of the magnetic flux present in each element. In FIG. 8C, the array of permanent magnetic elements can be spatially arranged so that the magnetization direction of each element can be tilted relative to each other by 90° in the direction of the longitudinal axis of the array, applying the Halbach principle. This spatial arrangement can preferentially augment the magnetic field along one side of the array, while cancelling the magnetic field to near zero on the other side. This leads to the desired effect in that the resulting magnetic field can be increased up to two-fold as strong along the preferential side in which the magnetic flux can be intentionally confined. In this manner, the spatial distribution of the magnetic elements of interest can be oriented during the manufacture of the magnetic accessory base that can exhibit enhanced magnetic fields/properties, and thereby, enabling strong/stable attachment of various magnetic personalizable accessories directly against (perpendicularly) the main surface of the pre-coated dermal adhesive film if desired. It is noted that the spatial distribution/orientation of the magnetic elements as shown in FIG. 8C results in a magnetic field that can be stronger than shown in FIGS. 8A and 8B.

As another embodiment, the exemplary spatial distribution/orientation of the magnetic elements as shown in FIGS. 8A-C can be modified into various configurations more suitable for the different shapes of the accessory base of interest, including circles, oblongs, squares, rectangles, stars, shells, flowers, among others.

### E. Exemplary Materials and Processes for Producing the Magnetic Accessory Base of Personalizable Accessories

The magnetically attachable personalizable accessory comprises: (a) a magnetic accessory base incorporating one or more magnetic elements exhibiting high magnetic coercivity; and (b) a visible accessory surface of interest, wherein the magnetic elements are positioned towards the surface of the accessory base capable of contacting and attaching the surface of a magnetic dermal adhesive film formed over a skin of interest. The materials and methods for producing suitable “magnetic accessory bases” and “magnetic elements” are further described below.

The “magnetic accessory base” can be manufactured from a wide range of polymeric materials, provided that these materials can be formed substantially flexible and conforming to the skin, have adequate dermal compatibility without significant irritation potential, and are easily blended with the contemplated magnetic elements. Suitable polymers capable of forming an accessory base include fluoroelastomers, polysulfones, polyamides, polyurethanes, polyesters, polyethers, silicones, polycarbonates, polyurethane carbonates, polyesters, polyamides, polyimides, polyvinyls, and polyolefins, polyvinyl alcohols, polyacetates, including blends and mixtures thereof.

Particularly suited for a skin-contacting application can be polyurethane and silicone-based materials, due to their intrinsically low irritation potential, good dermal compatibility and a widely adjustable range of flexibility. These polymer materials are available with a wide range of elastic properties, for example, that can be expressed through a measurement of shore hardness A, which can range from 0 to about 90 durometers. For the given application, ranges smaller than 50 durometers, more preferably smaller than 40 durometers and most preferably smaller than 30 durometers can be considered an optimum range of softness. In addition, the aforementioned polymers possess excellent compounding properties, and can be processed through a wide range of methods ideally suitable for blending with contemplated “magnetic elements.”

The magnetic elements are not limited as to the exact disposition of the polymer substrate utilized for forming the contemplated “magnetic accessory base.” For example, the magnetic elements can be incorporated, adhered, layered, reacted, blended/mixed, embedded, grafted, bonded, compounded, crosslinked, copolymerized and/or reacted with the monomers for forming the polymer substrate, or can be processed as part of an intermediate layer that can be adhered, adjoined, affixed and/or reacted, or combined with the polymer substrate utilized for forming the “magnetic accessory base” in any manner. Furthermore, the magnetic elements can be combined with a conventional polymer, and the combination can be adhered onto/around/within the “magnetic accessory base” or the skin-facing surface of the accessory so that the magnetic element and the polymer substrate can be deposited simultaneously or sequentially. The magnetic elements can be incorporated into the polymer substrate of the “magnetic accessory base” through various processes, including dispersion, suspension, sedimentation, encapsulation, coating, layering, film deposition, sputtering, spraying and similar functional equivalent. Depending on the particular process applied, the spatial distribution of the magnetic elements dispersed in the “magnetic accessory base” can be substantially isotropic, or anisotropic, homogeneous, or inhomogeneous, or in the form of a gradient or non-gradient distribution.

Because the magnetic adherent force between the surface of a first ferromagnetic material (e.g., magnetic element) and the surface of a second ferromagnetic material (e.g., magnetic particles) (towards each other) depends on the orientation and distance between them, the smaller the gap existing between the “magnetic elements” incorporated into the “magnetic accessory base” and the “magnetic particles” in the “magnetic dermal adhesive,” the stronger the resulting adherent force. Thus, the magnetic elements in the “magnetic accessory base” are preferably arranged in close local proximity to the surface of the dermal adhesive. To achieve this specific spatial arrangement, the magnetic particles can be sedimented by gravitation within the monomer solution during the curing process of the polymer substrate utilized for forming the “magnetic accessory base.” Alternatively, a magnetic force can be utilized to apply reverse gravitational pull on the magnetic elements when suspended in the monomer solution, such that the particles are preferably oriented not at the bottom, but at the top of the substrate.

The foregoing description, for purposes of explanation, refers to specific nomenclature to provide a thorough understanding of the invention. However, it will be apparent to persons skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously, many modifications and variations are possible in view of the above teachings. The embodiments are shown and described in order to best explain the principles of the invention and practical applications, to thereby enable others skilled in the art to best utilize the invention and various embodiments with various modifications as suitable for the particular uses contemplated. It is intended that the scope of the invention be defined by the following claims and their equivalents.

### EXAMPLES

### Example 1

Exemplary Structural Adhesive Formulation for Load-Bearing Magnetic Dermal Adhesives Employing Iron (II,III) Oxide Nanocrystals (SPIO)

As several embodiments, exemplary structural adhesive formulations capable of stably adhering to “a skin of interest” can include the following agents:

| | | Composition: | | |
|---|---|---|---|---|
| | | A | B | C |
| Function | Ingredient(s) | (w/w)% | (w/w)% | (w/w)% |
| Magnetic Element(s) | Iron (II, III) oxide nanocrystals (SPIO) | 35.0 | 40.0 | 45.0 |
| Adhesive agent(s) | Methacrylic acid-2-ethylhexyl acrylate | 10.0 | 15.0 | 20.0 |
| | copolymer | | | |
| Viscosifying agent(s) | Butyl acrylate/methyl | 20.0 | 15.0 | 10.0 |
| | methacrylate/methacrylic acid | | | |
| | copolymer | | | |
| Film forming agent(s) | 2-Propenoic acid, 2-methyl-, polymer | 20.0 | 15.0 | 10.0 |
| | with ethenylbenzene | | | |
| Gelling agent(s) | Xanthan gum / | 1.0 | 1.0 | 1.0 |
| | Cellulose / | | | |
| | Clay Mineral | | | |
| Emollient / | 1,3-Butylene Glycol | 2.9 | 2.9 | 2.9 |
| Humectant(s) | | | | |
| Surfactant(s) | Polyoxyethylene (20)-sorbitan- | 1.0 | 1.0 | 1.0 |
| | monolaurat / | | | |
| | Polyoxyethylene (40) ricinoleyl ether | | | |
| Preservative(s) | 2-Acetyl-5-hydroxy-3-oxo-4-hexenoic | 0.1 | 0.1 | 0.1 |
| | acid δ-lactone sodium salt / | | | |
| | (2E,4E)-hexa-2,4-dienoic acid / | | | |
| | 2-phenoxyethanol / | | | |
| | Undec-10-enoic acid | | | |
| Solvent(s) | Water | 10.0 | 10.0 | 10.0 |

### Example 2

### Exemplary Structural Adhesive Formulation for Load-Bearing Magnetic Dermal Adhesives Employing Iron (II,III) Oxide Nanopowder (>50-100 nm Particle Size)

As several embodiments, exemplary structural adhesive formulations capable of stably adhering to “a skin of interest” can include the following agents:

| | | Composition: | | |
|---|---|---|---|---|
| | | D | E | FC |
| Function | Ingredient(s) | (w/w)% | (w/w)% | (w/w)% |
| Magnetic Element(s) | Iron (II, III) oxide nanopowder, | 35.0 | 40.0 | 45.0 |
| | > 50-100 nm particle size | | | |
| Adhesive agent(s) | Methacrylic acid-2-ethylhexyl acrylate | 10.0 | 15.0 | 20.0 |
| | copolymer | | | |
| Viscosifying agent(s) | Butyl acrylate/methyl | 20.0 | 15.0 | 10.0 |
| | methacrylate/methacrylic acid | | | |
| | copolymer | | | |
| Film forming agent(s) | 2-Propenoic acid, 2-methyl-, polymer | 20.0 | 15.0 | 10.0 |
| | with ethenylbenzene | | | |
| Gelling agent(s) | Xanthan gum / | 1.0 | 1.0 | 1.0 |
| | Cellulose / | | | |
| | Clay Mineral | | | |
| Emollient / | 1,3-Butylene Glycol | 2.9 | 2.9 | 2.9 |
| Humectant(s) | | | | |
| Surfactant(s) | Polyoxyethylene (20)-sorbitan- | 1.0 | 1.0 | 1.0 |
| | monolaurat / | | | |
| | Polyoxyethylene (40) ricinoleyl ether | | | |
| Preservative(s) | 2-Acetyl-5-hydroxy-3-oxo-4-hexenoic | 0.1 | 0.1 | 0.1 |
| | acid δ-lactone sodium salt / | | | |
| | (2E,4E)-hexa-2,4-dienoic acid / | | | |
| | 2-phenoxyethanol / | | | |
| | Undec-10-enoic acid | | | |
| Solvent(s) | Water | 10.0 | 10.0 | 10.0 |

### Example 3

### Exemplary Structural Adhesive Formulation for Load-Bearing Magnetic Dermal Adhesives Employing Iron (II,III) Oxide Micronized Powder (<5 μm Particle Size)

As several embodiments, exemplary structural adhesive formulations capable of stably adhering to “a skin of interest” can include the following agents:

| | | Composition: | | |
|---|---|---|---|---|
| | | G | H | I |
| Function | Ingredient(s) | (w/w)% | (w/w)% | (w/w)% |
| Magnetic Element(s) | Iron (II, III) oxide micronized powder, | 35.0 | 40.0 | 45.0 |
| | < 5 μm particle size | | | |
| Adhesive agent(s) | Methacrylic acid-2-ethylhexyl acrylate | 10.0 | 15.0 | 20.0 |
| | copolymer | | | |
| Viscosifying agent(s) | Butyl acrylate/methyl | 20.0 | 15.0 | 10.0 |
| | methacrylate/methacrylic acid | | | |
| | copolymer | | | |
| Film forming agent(s) | 2-Propenoic acid, 2-methyl-, polymer | 20.0 | 15.0 | 10.0 |
| | with ethenylbenzene | | | |
| Gelling agent(s) | Xanthan gum / | 1.0 | 1.0 | 1.0 |
| | Cellulose / | | | |
| | Clay Mineral | | | |
| Emollient / | 1,3-Butylene Glycol | 2.9 | 2.9 | 2.9 |
| Humectant(s) | | | | |
| Surfactant(s) | Polyoxyethylene (20)-sorbitan- | 1.0 | 1.0 | 1.0 |
| | monolaurat / | | | |
| | Polyoxyethylene (40) ricinoleyl ether | | | |
| Preservative(s) | 2-Acetyl-5-hydroxy-3-oxo-4-hexenoic | 0.1 | 0.1 | 0.1 |
| | acid δ-lactone sodium salt / | | | |
| | (2E,4E)-hexa-2,4-dienoic acid / | | | |
| | 2-phenoxyethanol / | | | |
| | Undec-10-enoic acid | | | |
| Solvent(s) | Water | 10.0 | 10.0 | 10.0 |

### Example 4

### Exemplary Structural Adhesive Formulation for Load-Bearing Magnetic Dermal Adhesives Employing Neodymium-Iron-Boron Nd₂Fe₁₄B Powder (>50-60 μm Particle Size)

As several embodiments, exemplary structural adhesive formulations capable of stably adhering to “a skin of interest” can include the following agents:

| | | Composition: | | |
|---|---|---|---|---|
| | | J | K | L |
| Function | Ingredient(s) | (w/w)% | (w/w)% | (w/w)% |
| Magnetic Element(s) | Neodymium-iron-boron Nd2Fe14B | 35.0 | 40.0 | 45.0 |
| | powder, > 50-60 μm particle size | | | |
| Adhesive agent(s) | Methacrylic acid-2-ethylhexyl | 10.0 | 15.0 | 20.0 |
| | acrylate copolymer | | | |
| Viscosifying agent(s) | Butyl acrylate/methyl | 20.0 | 15.0 | 10.0 |
| | methacrylate/methacrylic acid | | | |
| | copolymer | | | |
| Film forming agent(s) | 2-Propenoic acid, 2-methyl-, polymer | 20.0 | 15.0 | 10.0 |
| | with ethenylbenzene | | | |
| Gelling agent(s) | Xanthan gum / | 1.0 | 1.0 | 1.0 |
| | Cellulose / | | | |
| | Clay Mineral | | | |
| Emollient / | 1,3-Butylene Glycol | 2.9 | 2.9 | 2.9 |
| Humectant(s) | | | | |
| Surfactant(s) | Polyoxyethylene (20)-sorbitan- | 1.0 | 1.0 | 1.0 |
| | monolaurat / | | | |
| | Polyoxyethylene (40) ricinoleyl ether | | | |
| Preservative(s) | 2-Acetyl-5-hydroxy-3-oxo-4-hexenoic | 0.1 | 0.1 | 0.1 |
| | acid δ-lactone sodium salt / | | | |
| | (2E,4E)-hexa-2,4-dienoic acid / | | | |
| | 2-phenoxyethanol / | | | |
| | Undec-10-enoic acid | | | |
| Solvent(s) | Water | 10.0 | 10.0 | 10.0 |

### Example 5

### Exemplary Structural Adhesive Formulation for Load-Bearing Magnetic Dermal Adhesives Employing Barium Ferrite BaFe₁₂O₁₉Powder (>40-50 μm Particle Size)

As several embodiments, exemplary structural adhesive formulations capable of stably adhering to “a skin of interest” can include the following agents:

| | | Composition: | | |
|---|---|---|---|---|
| | | J | K | L |
| Function | Ingredient(s) | (w/w)% | (w/w)% | (w/w)% |
| Magnetic Element(s) | Barium ferrite BaFe12019 powder, | 35.0 | 40.0 | 45.0 |
| | > 40-50 μm particle size | | | |
| Adhesive agent(s) | Methacrylic acid-2-ethylhexyl | 10.0 | 15.0 | 20.0 |
| | acrylate copolymer | | | |
| Viscosifying agent(s) | Butyl acrylate/methyl | 20.0 | 15.0 | 10.0 |
| | methacrylate/methacrylic acid | | | |
| | copolymer | | | |
| Film forming agent(s) | 2-Propenoic acid, 2-methyl-, polymer | 20.0 | 15.0 | 10.0 |
| | with ethenylbenzene | | | |
| Gelling agent(s) | Xanthan gum / | 1.0 | 1.0 | 1.0 |
| | Cellulose / | | | |
| | Clay Mineral | | | |
| Emollient / | 1,3-Butylene Glycol | 2.9 | 2.9 | 2.9 |
| Humectant(s) | | | | |
| Surfactant(s) | Polyoxyethylene (20)-sorbitan- | 1.0 | 1.0 | 1.0 |
| | monolaurat / | | | |
| | Polyoxyethylene (40) ricinoleyl ether | | | |
| Preservative(s) | 2-Acetyl-5-hydroxy-3-oxo-4-hexenoic | 0.1 | 0.1 | 0.1 |
| | acid δ-lactone sodium salt / | | | |
| | (2E,4E)-hexa-2,4-dienoic acid / | | | |
| | 2-phenoxyethanol / | | | |
| | Undec-10-enoic acid | | | |
| Solvent(s) | Water | 10.0 | 10.0 | 10.0 |

1-19. (canceled)

20. A magnetically attachable personalizable accessory system, comprising:
(a) a curable, load-bearing magnetic dermal adhesive adherable to a skin surface and incorporating one or more magnetic particles compatible with a curable, load-bearing structural adhesive; and
(b) a magnetic personalizable accessory incorporating one or more magnetic elements exhibiting high intrinsic coercivity and positioned at least at the base of the magnetic personalizable accessory,
wherein the adhesive force between the cured, load-bearing structural adhesive of the skin-adhered magnetic dermal adhesive and the skin surface exceeds the magnetic force generated between the cured, load-bearing magnetic dermal adhesive and the magnetic personalizable accessory,
wherein the magnetic force between the cured, load-bearing magnetic dermal adhesive and the magnetic personalizable accessory exceeds the gravitational force acting on the magnetic personalizable accessory, and
whereby the magnetic interaction between the magnetic particles incorporated into the cured, load-bearing magnetic dermal adhesive and the magnetic elements incorporated into the magnetic personalizable accessory stably attaches the magnetic personalizable accessory to the skin surface.

21. The magnetically attachable personalizable accessory system of claim 20, wherein the curable, load-bearing magnetic dermal adhesive comprises (a) the one or more magnetic particles suitable for use in a cosmetic formulation, and (b) the curable, load-bearing structural adhesive, capable of adhering to the skin surface, and capable of magnetically supporting the attachment of the magnetic personalizable accessory.

22. The magnetically attachable personalizable accessory system of claim 20, wherein the one or more magnetic particles are ferromagnetic compounds selected from the group consisting of iron oxides, hematite Fe₂O₃, magnetite Fe^{II}(Fe^{III})₂O₄, super-paramagnetic iron oxides, chromium dioxide CrO₂, barium ferrites, bismuth ferrite BiFeO₃, manganese-zinc ferrite MnZn, cobalt ferrite CoFe₂O₄, strontium ferrite SrFe₁₂O₁₉, and yttrium iron granate Y₃Fe₅O₁₂.

23. The magnetically attachable personalizable accessory system of claim 20, wherein the one or more magnetic elements are formed from one or more hard ferromagnetic materials.

24. The magnetically attachable personalizable accessory system of claim 23, wherein the one or more hard ferromagnetic materials are selected from the group consisting of bismanol and AlNiCo.

25. The magnetically attachable personalizable accessory system of claim 23, wherein the one or more hard ferromagnetic materials comprise one or more rare-earth compounds.

26. The magnetically attachable personalizable accessory system of claim 25, wherein the one or more rare-earth compounds comprise neodymium-iron-boron Nd₂Fe₁₄B.

27. The magnetically attachable personalizable accessory of claim 20, wherein the curable, load-bearing structural adhesive comprises a fluorinated acrylate polymer.

28. The magnetically attachable personalizable accessory system of claim 20, wherein the one or more magnetic elements in the magnetic personalizable accessory are arranged to form a one-sided flux distribution.

29. A method for making a magnetically attachable personalizable accessory system of claim 20, the method comprising:
(a) providing the curable, load-bearing magnetic dermal adhesive incorporating the one or more magnetic particles compatible with the curable, load-bearing structural adhesive adherable to the skin surface; and
(b) providing the magnetic personalizable accessory incorporating the one or more magnetic elements exhibiting high intrinsic coercivity,
wherein the adhesive force between the cured, load-bearing structural adhesive of the skin-adhered magnetic dermal adhesive and the skin surface exceeds the magnetic force generated between the cured, load-bearing magnetic dermal adhesive and the magnetic personalizable accessory,
wherein the magnetic force between the cured, load-bearing magnetic dermal adhesive and the magnetic personalizable accessory exceeds the gravitational force acting on the magnetic personalizable accessory, and
whereby stably attaching the magnetic personalizable accessory to the skin surface due to the interaction between the one or more magnetic particles incorporated into the cured, load-bearing magnetic dermal adhesive and the one or more magnetic elements incorporated into the magnetic personalizable accessory.

30. A method of using a magnetically attachable personalizable accessory system of claim 20, comprising:
exposing the curable, load-bearing magnetic dermal adhesive onto the skin surface under ambient conditions to allow the curing process to occur within the curable, load-bearing magnetic dermal adhesive to form a stable adhesive film over the skin surface; and
positioning and attaching the magnetic personalizable accessory over the cured, load-bearing magnetic dermal adhesive film stably adhered over the skin surface.

31-35. (canceled)

36. The magnetically attachable personalizable accessory system of claim 20, wherein the base of the magnetic personalizable accessory comprises at least one polymer selected from the group consisting of fluoroelastomers, polysulfones, polyamides, polyurethanes, polyesters, polyethers, silicones, polycarbonates, polyurethane carbonates, polyesters, polyamides, polyimides, polyvinyls, polyolefins, polyvinyl alcohols, polyacetates, and mixtures thereof.

37. The magnetically attachable personalizable accessory system of claim 20, wherein the base of the magnetic personalizable accessory comprises polyurethanes.

38. The magnetically attachable personalizable accessory system of claim 20, wherein the curable, load-bearing structural adhesive comprises at least one acrylic polymer selected from the group consisting of styrene-methacrylic acid copolymers, styrene-acrylate copolymers, ethylhexyl acrylate-acrylate copolymers, acrylate-acrylate copolymers, and mixtures or blends thereof.

39. The magnetically attachable personalizable accessory system of claim 20, wherein the at least one acrylic copolymer is selected from the group consisting of a butyl acrylate-ethylhexyl acrylate copolymer, a methyl methacrylate-ethylhexyl acrylate copolymer, a methacrylic acid-ethylhexyl acrylate copolymer, a butyl acrylate-styrene copolymer, a methyl methacrylate-styrene copolymer, a methacrylic acid-styrene copolymer, and mixtures or blends thereof.

40. The magnetically attachable personalizable accessory system of claim 20, wherein the curable, load-bearing structural adhesive further comprises phenoxyethanol.
